(19) United States
(12) Patent Application Publication
(10) Pub. No.: US 2015/0057351 A1
(43) Pub. Date: Feb. 26, 2015

(54) METHOD FOR TREATING NEUROPATHIC PAIN

(71) Applicant: SUCAMPO AG, Zug (CH)

(72) Inventor: Ryuji UENO, Saint Michaels, MD (US)

(73) Assignee: SUCAMPO AG, Zug (CH)

(21) Appl. No.: 14/464,232

(22) Filed: Aug. 20, 2014

Related U.S. Application Data
(60) Provisional application No. 61/868,750, filed on Aug. 22, 2013.
(60) Provisional application No. 61/918,124, filed on Dec. 19, 2013.

(51) Int. Cl. C07C 57/26; C07C 53/134

(52) U.S. Cl. CPC C07C 57/26; C07C 53/134
USPC 514/559

(57) ABSTRACT

A method for treating neuropathic pain in a mammalian subject, which comprises administering an effective amount of a fatty acid derivative, is provided.

### CROSS REFERENCE TO RELATED APPLICATIONS

This application claims the benefit of U.S. Provisional Patent Application No. 61/868,750 filed Aug. 22, 2013 and U.S. Provisional Patent Application No. 61/918,124 filed Dec. 19, 2013, the entire contents of which are incorporated by reference herein.

### TECHNICAL FIELD

The present invention relates to use of a fatty acid derivative for treating neuropathic pain in a mammalian subject.

### BACKGROUND

Neuropathic pain is a complex, chronic pain state that usually is accompanied by tissue injury. With neuropathic pain, the nerve fibers themselves may be damaged, dysfunctional, or injured. These damaged nerve fibers send incorrect signals to other pain centers. The impact of nerve fiber injury includes a change in nerve function both at the site of injury and areas around the injury.

One example of neuropathic pain is called phantom limb syndrome. This rare condition occurs when an arm or a leg has been removed because of illness or injury, but the brain still gets pain messages from the nerves that originally carried impulses from the missing limb. These nerves now misfire and cause pain.

Neuropathic pain often seems to have no obvious cause; but, some common causes of neuropathic pain include: Alcoholism, Amputation, Back, leg, and hip problems, Chemotherapy, Diabetes, Facial nerve problems, HIV infection or AIDS, Multiple sclerosis, Shingles, Spine surgery. Neuropathic pain symptoms may include: Shooting and Tingling.

Some neuropathic pain studies suggest the use of non-steroidal anti-inflammatory drugs, such as Aleve or Motrin, may ease pain. Some people may require a stronger painkiller, such as those containing morphine. Anticonvulsant and antidepressant drugs seem to work in some cases.

If another condition, such as diabetes, is involved, better management of that disorder may alleviate the pain. Effective management of the condition can also help prevent further nerve damage.

In cases that are difficult to treat, a pain specialist may use an invasive or implantable device to effectively manage the pain. Electrical stimulation of the nerves involved in neuropathic pain may significantly control the pain symptoms.

Other kinds of treatments can also help with neuropathic pain. Some of these include: Physical therapy, Working with a counselor, Relaxation therapy, Massage therapy, Acupuncture.

Unfortunately, neuropathic pain often responds poorly to standard pain treatments and occasionally may get worse instead of better over time. For some people, it can lead to serious disability.
(http://www.webmd.com/pain-management/guide/neuropathic-pain)

Fatty acid derivatives are members of class of organic carboxylic acids, which are contained in tissues or organs of human or other mammals, and exhibit a wide range of physiological activity. Some fatty acid derivatives found in nature generally have a prostanoic acid skeleton as shown in the formula (A):

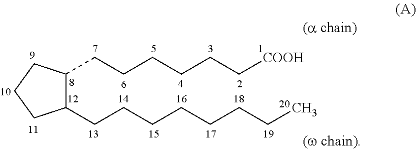 [SMILES: CCCCCCCCC1CCC[C@@H]1CCCCCCC(=O)O]

On the other hand, some of synthetic prostaglandin (PG) analogues have modified skeletons. The primary PGs are classified into PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-,and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGs are known to have various pharmacological and physiological activities, for example, vasodilatation, inducing of inflammation, platelet aggregation, stimulating uterine muscle, stimulating intestinal muscle, anti-ulcer effect and the like.

Prostones, having an oxo group at position 15 of prostanoic acid skeleton (15-keto type) and having a single bond between positions 13 and 14 and an oxo group at position 15 (13,14-dihydro-15-keto type), are fatty acid derivatives known as substances naturally produced by enzymatic actions during metabolism of the primary PGs and have some therapeutic effect.

U.S. Pat. No. 8,202,909 to Ueno describes the specific prostaglandin compounds are useful for treating central nervous system disorders, and U.S. Pat. No. 8,143,316 to Ueno describes the specific prostaglandin compounds are useful for treating peripheral vascular diseases.

However it is not known how the fatty acid derivatives act on the neuropathic pain which is a complex, chronic pain state, often seems to have no obvious cause, and often responds poorly to standard pain treatments.

### DISCLOSURE OF THE INVENTION

The present invention relates to a method for treating neuropathic pain in a mammalian subject, which comprises administering to the subject in need thereof an effective amount of a fatty acid derivative represented by the formula (I):

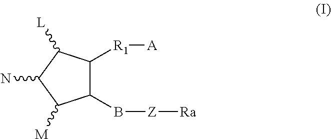

wherein L, M and N are hydrogen, hydroxy, halogen, lower alkyl, hydroxy(lower)alkyl, lower alkanoyloxy or oxo, wherein the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

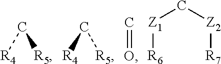

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

### DETAILED DESCRIPTION OF THE INVENTION

The nomenclature of the fatty acid derivative used herein is based on the numbering system of the prostanoic acid represented in the above formula (A).

The formula (A) shows a basic skeleton of the C-20 fatty acid derivative, but the present invention is not limited to those having the same number of carbon atoms. In the formula (A), the numbering of the carbon atoms which constitute the basic skeleton of the fatty acid derivatives starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of carboxy group (C-1). Similarly, when the number of carbon atoms is decreased in the ω-chain, the number is deleted in the order starting from position 20; and when the number of carbon atoms is increased in the ω-chain, the carbon atoms at the position or later are named as a substituent at position 20. Stereochemistry of the compounds is the same as that of the above formula (A) unless otherwise specified.

In general, each of PGD, PGE and PGF represents a fatty acid derivative having hydroxy groups at positions 9 and/or 11, but in the present specification they also include those having substituents other than the hydroxy groups at positions 9 and/or 11. Such compounds are referred to as 9-deoxy-9-substituted-fatty acid derivatives or 11-deoxy-11-substituted-fatty acid derivatives. A fatty acid derivative having hydrogen in place of the hydroxy group is simply named as 9- or 11-deoxy-fatty acid derivative.

As stated above, the nomenclature of a fatty acid derivative is based on the prostanoic acid skeleton. In the case the compound has similar partial structure as the primary PG, the abbreviation of “PG” may be used. Thus, a fatty acid derivative whose α-chain is extended by two carbon atoms, that is, having 9 carbon atoms in the α-chain is named as 2-decarboxy-2-(2-carboxyethyl)-PG compound. Similarly, a fatty acid derivative having 11 carbon atoms in the α-chain is named as 2-decarboxy-2-(4-carboxybutyl)-PG compound. Further, a fatty acid derivative whose ω-chain is extended by two carbon atoms, that is, having 10 carbon atoms in the ω-chain is named as 20-ethyl-PG compound. These compounds, however, may also be named according to the IUPAC nomenclatures.

Examples of the analogues including substitution compounds or derivatives of the above described fatty acid derivative include a fatty acid derivative whose carboxy group at the end of the alpha chain is esterified; a fatty acid derivative whose α chain is extended, a physiologically acceptable salt thereof, a fatty acid derivative having a double bond between positions 2 and 3 or a triple bond between positions 5 and 6; a fatty acid derivative having substituent(s) on carbon atom(s) at position(s) 3, 5, 6, 16, 17, 18, 19 and/or 20; and a fatty acid derivative having a lower alkyl or a hydroxy(lower)alkyl group at position 9 and/or 11 in place of the hydroxy group.

According to the present invention, preferred substituents on the carbon atom at position(s) 3, 17, 18 and/or 19 include alkyl having 1-4 carbon atoms, especially methyl and ethyl. Preferred substituents on the carbon atom at position 16 include lower alkyls such as methyl and ethyl, hydroxy, halogen atom such as chlorine and fluorine, and aryloxy such as trifluoromethylphenoxy. Preferred substituents on the carbon atom at position 17 include lower alkyl such as methyl and ethyl, hydroxy, halogen atom such as chlorine and fluorine, and aryloxy such as trifluoromethylphenoxy. Preferred substituents on the carbon atom at position 20 include saturated or unsaturated lower alkyl such as C₁₋₄ alkyl, lower alkoxy such as C₁₋₄ alkoxy, and lower alkoxy alkyl such as C₁₋₄ alkoxy-C₁₋₄ alkyl. Preferred substituents on the carbon atom at position 5 include halogen atoms such as chlorine and fluorine. Preferred substituents on the carbon atom at position 6 include an oxo group forming a carbonyl group. Stereochemistry of PGs having hydroxy, lower alkyl or hydroxy(lower)alkyl substituent on the carbon atom at positions 9 and 11 may be α, β or a mixture thereof.

Further, the above described analogues or derivatives may have a co chain shorter than that of the primary PGs and a substituent such as alkoxy, cycloalkyl, cycloalkyloxy, phenoxy and phenyl at the end of the truncated ω-chain.

A fatty acid derivative used in the present invention is represented by the formula (I):

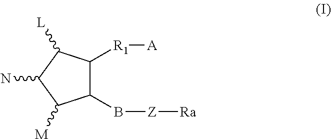

wherein L, M and N are hydrogen, hydroxy, halogen, lower alkyl, hydroxy(lower)alkyl, lower alkanoyloxy or oxo, wherein the five-membered ring may have at least one double bond;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

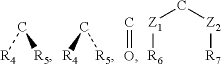

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

A preferred compound used in the present invention is represented by the formula (II):

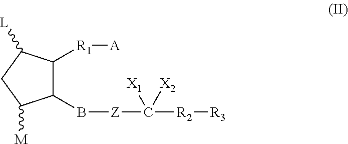

wherein L and M are hydrogen atom, hydroxy, halogen, lower alkyl, hydroxy(lower)alkyl, lower alkanoyloxy or oxo, wherein the five-membered ring may have one or more double bonds;

A is —CH₃, or —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

Z is

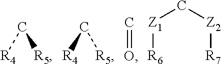

or single bond

wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₂ and Z₂ are oxygen, nitrogen or sulfur; R₆ and R₇ are optionally substituted lower alkyl, which is optionally linked together to form lower alkylene;

X₂ and X₂ are hydrogen, lower alkyl, or halogen;

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur;

R₂ is a single bond or lower alkylene; and

R₃ is lower alkyl, lower alkoxy, lower alkanoyloxy, cyclo(lower)alkyl, cyclo(lower)alkyloxy, aryl, aryloxy, heterocyclic group or heterocyclic-oxy group, and at least one of carbon atom in the aliphatic hydrocarbon is optionally substituted by oxygen, nitrogen or sulfur.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O-, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above. The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy(lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy(lower)alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A mean a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydrogen, hydroxy and oxo, and especially, L and M are both hydroxy, or L is oxo and M is hydrogen or hydroxy.

Preferred example of A is —COOH, its pharmaceutically acceptable salt, ester or amide thereof.

Preferred example of X₁ and X₂ are both being hydrogens or halogen atoms, and in case of halogen atoms, more preferably, fluorine atoms, so called 16,16-difluoro type.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur. Examples of R₁ include, for example, the following groups:

—CH₂—CH₂—CH₂—CH₂—CH₂—CH₂—,

—CH₂—CH=CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—O—CH₂—,

—CH₂—CH═CH—CH₂—O—CH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂—,

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—C≡C—CH₂—CH₂—CH₂—CH₂—CH₂—, and

—CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains having one carbon atom. Further, at least one carbon atom in the aliphatic hydrocarbon is optionally substituted by oxygen, nitrogen or sulfur.

In embodiments of the present invention, representative compounds of the formula (I) or (II) include compounds of the formula (I) wherein Ra is substituted by halogen and/or Z is C═O;
compounds of the formula (II) wherein one of X₁ and X₂ is substituted by halogen and/or Z is C═O;
compounds of the formula (II) wherein L is ═O or —OH, M is H or OH, A is COOH or a functional derivative thereof, B is —CH₂—CH₂—, Z is C═O, X₁ is halogen (e.g. X₁ is Cl, Br, I or F) or hydrogen, X₂ is halogen (e.g. X₂ is Cl, Br, I or F) or hydrogen, R₁ is a saturated or unsaturated bivalent straight C₆ aliphatic hydrocarbon residue, R₂ is a single bond, and R₃ is straight or branched lower alkyl (e.g. C₄₋₆ alkyl) optionally substituted by oxygen, nitrogen or sulfur;
compounds of the formula (II) wherein L is ═O, M is OH, A is COOH or a functional derivative thereof, B is —CH₂—CH₂—, Z is C═O, X₁ is halogen (e.g. X₁ is Cl, Br, I or F) or hydrogen, X₂ is halogen (e.g. X₂ is Cl, Br, I or F) or hydrogen, R₁ is a saturated or unsaturated bivalent straight C₆ aliphatic hydrocarbon residue, R₂ is a single bond, and R₃ is straight or branched lower alkyl optionally substituted by oxygen, nitrogen or sulfur;
compounds of the formula (II) wherein L is ═O, M is OH, A is COOH or a functional derivative thereof, B is —CH₂—CH₂—, Z is C═O, X₂ and X₂ are halogen atoms (e.g. X₂ and X₂ are Cl, Br, I or F), R₁ is a saturated or unsaturated bivalent straight C₆ aliphatic hydrocarbon residue, R₂ is a single bond, and R₃ is straight or branched lower alkyl (e.g. C₄ alkyl or C₅ alkyl);
compounds of the formula (II) wherein L is ═O, M is OH, A is COOH or a functional derivative thereof, B is —CH₂—CH₂—, Z is C═O, X₂ and X₂ are fluorine atoms, R₁ is a saturated or unsaturated bivalent straight C₆ aliphatic hydrocarbon residue, R₂ is a single bond, and R₃ is straight or branched lower alkyl (e.g. C₄ alkyl or C₅ alkyl);
compounds of the formula (II) wherein L is ═O, M is H or OH, A is COOH or a functional derivative thereof, B is —CH₂—CH₂—, Z is C═O, X₁ and X₂ are halogen atoms (e.g. X₁ and X₂ are Cl, Br, I or F), R₁ is a saturated or unsaturated bivalent straight C₆ aliphatic hydrocarbon residue, R₂ is a single bond, and R₃ is —CH₂—CH₂—CH₂—CH₃ or —CH₂—CH(CH₃)—CH₂—CH₃ .

In further embodiment, representative compounds used in the present invention include (−)-7-[(2R,4aR,5R,7aR)-2-(1,1-difluoropentyl)-2-hydroxy-6-oxooctahydrocyclopenta[b]pyran-5-yl]heptanoic acid (lubiprostone), (−)-7-{(2R,4aR,5R,7aR)-2-[(3S)-1,1-difluoro-3-methylpentyl]-2-hydroxy-6-oxooctahydrocyclopenta[b]pyran-5-yl}heptanoic acid (cobiprostone), (+)-isopropyl (Z)-7-[(1R,2R,3R,5S)-3,5-dihydroxy-2-(3-oxodecyl)cyclopentyl]hept-5-enoate (isopropyl unoprostone) (−)-7-[(1R,2R)-2-(4,4-difluoro-3-oxooctyl)-5-oxocyclopentyl]heptanoic acid, (E)-7-[(1R,2R)-2-(4,4-difluoro-3-oxooctyl)-5-oxocyclopentyl]hept-2-enoic acid, an isomer (including tautomeric isomer) thereof and a functional derivative thereof.

Preferably, (−)-7-[(1R,2R)-2-(4,4-difluoro-3-oxooctyl)-5-oxocyclopentyl]heptanoic acid or (E)-7-[(1R,2R)-2-(4,4-difluoro-3-oxooctyl)-5-oxocyclopentyl]hept-2-enoic acid may be used for the present invention.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

In the present invention, the fatty acid derivative which is dihydro between 13 and 14, and keto(═O) at 15 position may be in the keto-hemiacetal equilibrium by formation of a hemiacetal between hydroxy at position 11 and keto at position 15.

For example, it has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the present invention includes both isomers.

Further, the fatty acid derivatives used in the invention include the bicyclic compound and analogs or derivatives thereof.

The bicyclic compound is represented by the formula (III)

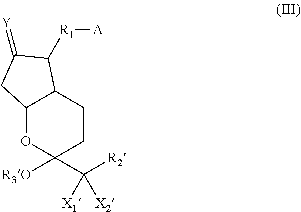

wherein, A is —CH₃, or —CH₂OH, —COCH₂OH, —COOH or a functional derivative thereof;

X₁′ and X₂′ are hydrogen, lower alkyl, or halogen;

Y is

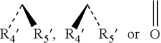

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated divalent lower or medium aliphatic hydrocarbon residue, which is unsubstituted or substituted with halogen, alkyl, hydroxy, oxo, aryl or heterocyclic group, and at least one of carbon atom in the aliphatic hydrocarbon is optionally substituted by oxygen, nitrogen or sulfur; and

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

R₃′ is hydrogen, lower alkyl, cyclo(lower)alkyl, aryl or heterocyclic group.

Furthermore, while the compounds used in the invention may be represented by a formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used in the same purpose. Some of the compounds used in the present invention may be prepared by the method disclosed in U.S. Pat. Nos. 5,073,569, 5,166,174, 5,221,763, 5,212,324, 5,739,161, 6,242,485 and 8,202,909 (these cited references are herein incorporated by reference).

The mammalian subject may be any mammalian subject including a human. The compound may be applied systemically or topically. Usually, the compound may be administered by oral administration, intranasal administration, inhalational administration, intravenous injection (including infusion), subcutaneous injection, ocular topical administration, intra rectal administration, intra vaginal administration, transdermal administration and the like.

The dose may vary depending on the strain of the animal, age, body weight, symptom to be treated, desired therapeutic effect, administration route, term of treatment and the like. A satisfactory effect can be obtained by systemic administration 1-4 times per day or continuous administration (e.g. repeated administration) at the amount of 0.00001-500mg/kg per day, more preferably 0.0001-100mg/kg. For example, 10-200 pg/day (e.g. 30, 60, 120 pg/day) of the compound disclosed herein (e.g. Compound A used in the Examples) may be administered to human (e.g. by injection). The compound disclosed herein may be repeatedly administered (e.g. for 2 weeks).

The compound may preferably be formulated in a pharmaceutical composition suitable for administration in a conventional manner. The composition may be those suitable for oral administration, intranasal administration, ocular topical administration, inhalational administration, injection or perfusion as well as it may be an external agent, suppository or pessary.

The composition of the present invention may further contain physiologically acceptable additives. Said additives may include the ingredients used with the present compounds such as excipient, diluent, filler, resolvent, lubricant, adjuvant, binder, disintegrator, coating agent, cupsulating agent, ointment base, suppository base, aerozoling agent, emulsifier, dispersing agent, suspending agent, thickener, tonicity agent, buffering agent, soothing agent, preservative, antioxidant, corrigent, flavor, colorant, a functional material such as cyclodextrin and biodegradable polymer, stabilizer. The additives are well known to the art and may be selected from those described in general reference books of pharmaceutics.

The amount of the above-defined compound in the composition of the invention may vary depending on the formulation of the composition, and may generally be 0.000001-10.0%, more preferably 0.00001-5.0%, most preferably 0.0001-1%. For example, 10-200 μg (e.g. 30, 60, 120 μg) of the compound disclosed herein (e.g. Compound A used in the Examples) may be contained in the composition of the invention.

Examples of solid compositions for oral administration include tablets, troches, sublingual tablets, capsules, pills, powders, granules and the like. The solid composition may be prepared by mixing one or more active ingredients with at least one inactive diluent. The composition may further contain additives other than the inactive diluents, for example, a lubricant, a disintegrator and a stabilizer. Tablets and pills may be coated with an enteric or gastroenteric film, if necessary. They may be covered with two or more layers. They may also be adsorbed to a sustained release material, or microcapsulated. Additionally, the compositions may be capsulated by means of an easily degradable material such gelatin. They may be further dissolved in an appropriate solvent such as fatty acid or its mono, di or triglyceride to be a soft capsule. Sublingual tablet may be used in need of fast-acting property.

Examples of liquid compositions for oral administration include emulsions, solutions, suspensions, syrups and elixirs and the like. Said composition may further contain a conventionally used inactive diluents e.g. purified water or ethyl alcohol. The composition may contain additives other than the inactive diluents such as adjuvant e.g. wetting agents and suspending agents, sweeteners, flavors, fragrance and preservatives.

The composition of the present invention may be in the form of spraying composition, which contains one or more active ingredients and may be prepared according to a known method.

Example of the intranasal preparations may be aqueous or oily solutions, suspensions or emulsions comprising one or more active ingredient. For the administration of an active ingredient by inhalation, the composition of the present invention may be in the form of suspension, solution or emulsion which can provide aerosol or in the form of powder suitable for dry powder inhalation. The composition for inhalational administration may further comprise a conventionally used propellant.

Examples of the injectable compositions of the present invention for parenteral administration include sterile aqueous or non-aqueous solutions, suspensions and emulsions. Diluents for the aqueous solution or suspension may include, for example, distilled water for injection, physiological saline and Ringer's solution.

Non-aqueous diluents for solution and suspension may include, for example, propylene glycol, polyethylene glycol, vegetable oils such as olive oil, alcohols such as ethanol and polysorbate. The composition may further comprise additives such as preservatives, wetting agents, emulsifying agents, dispersing agents and the like. They may be sterilized by filtration through, e.g. a bacteria-retaining filter, compounding with a sterilizer, or by means of gas or radioisotope irradiation sterilization. The injectable composition may also be provided as a sterilized powder composition to be dissolved in a sterilized solvent for injection before use.

The present external agent includes all the external preparations used in the fields of dermatology and otolaryngology, which includes ointment, cream, lotion and spray.

Another form of the present invention is suppository or pessary, which may be prepared by mixing active ingredients into a conventional base such as cacao butter that softens at body temperature, and nonionic surfactants having suitable softening temperatures may be used to improve absorbability.

According to the present invention, the fatty acid derivatives of the present invention are useful for treating neuropathic pain and its symptom in a mammalian subject, which comprises administering to the subject in need thereof. Especially, the effects of the fatty acid derivatives of the present invention increase by repeated administration, for example, 2 days administration, 4 days administration or 7 days administration and sustain the effect after stopping the administration and administration of consecutive days is better than single day treatment. Accordingly, the fatty acid derivatives of the present invention have also superiority on treating neuropathic pain with repeated administration. Furthermore, the fatty acid derivatives of the present invention improve pain-associated quality of life.

The term “neuropathic pain” used herein includes any neuropathic pains and the symptoms derived from any causes or no obvious causes. Some common causes of neuropathic pain include, but not limited to: alcoholism, amputation (including phantom limb syndrome), back (e.g. low back), limbs (e.g. lower limbs), and hip problems including arthritis such as osteoarthritis, rheumatoid arthritis and osteoporosis, cancer, chemotherapy (e.g. anti-cancer agent-induced), diabetes, facial nerve problems, Herpes zoster, HIV infection or AIDS, multiple sclerosis, shingles, spine surgery (e.g. post-spinal cord injury). Neuropathic pain symptoms may also include, but not limited to: shooting pain, tingling pain, gripping pain, hyperalgesia and allodynia. As described above, neuropathic pain often seems to have no obvious cause. The present invention also includes neuropathic pains without organic change by MRI, CT, X ray or other examination.

The term “treating” or “treatment” used herein includes prophylactic and therapeutic treatment, and any means of control such as prevention, care, relief of the condition, attenuation of the condition, arrest of progression, etc.

The pharmaceutical composition of the present invention may contain a single active ingredient or a combination of two or more active ingredients, as far as they are not contrary to the objects of the present invention.

In a combination of plural active ingredients, their respective contents may be suitably increased or decreased in consideration of their therapeutic effects and safety.

The term “combination” used herein means two or more active ingredient are administered to a patient simultaneously in the form of a single entity or dosage, or are both administered to a patient as separate entities either simultaneously or sequentially with no specific time limits, wherein such administration provides therapeutically effective levels of the two components in the body, preferably at the same time.

The present invention will be described in detail with reference to the following example, which, however, is not intended to limit the scope of the present invention.

### EXAMPLE 1

Male CD(SD) rats at 8 weeks old were used to prepare a neuropathic pain model. Rats were anesthetized with pentobarbital sodium, and the back skins were cut open along the median line from L₃ to S₁ (from the thoracic spine to the sacral vertebrae). The muscle layer along the apophysis was cut open from L₄ to L₆ on the vertebrae. The muscles in the surroundings of the vertebrae of L₄ to L₆ were shaved off with a bone chisels, and the ventralis apophysis on L₄-L₆ was excised with a bone rongeur forceps. Left sides of the ventralis (vertebral arch) of L₄ and L₅ were excised with bone rongeur forceps, and then each nerve root was exposed. The nerve roots of L₄ and L₅ were separated from the vertebrae using micro dissecting tweezers. Each nerve root was lightly ligated with a sterilized silk suture (No. 5-0). Muscle layer was closed with a sterilized nylon suture (No. 4-0) at 3-5 places. Then, the back skin was closed with a sterilized nylon suture (No. 4-0). The animals were observed on their general health condition and body weight for 4 days after the operation. At 4 days after the model preparation surgery, the animal's pain threshold in the plantar surface of the model paw was measured using a Dynamic Planter Aesthesiometer (37400, Ugo Basile), i.e. mechanical pressure stimulation, which was gradually increased from 0 to 30 g during 40 seconds, was applied to the plantar surface of the animal until the animal escapes from the stimulation. Animals with a pain threshold of more than 8.0 g in the model paw were excluded from the experiment. Test substances were intravenously administered to the model animals twice a day for 7 days from 5 days after the surgical operation. The pain threshold was measured before the start of the administration, i.e. 4 days after the model preparation surgery, and 2-, 4-, 7- and 10-day after the start of administration. Measurement of pain threshold was carried out 30 minutes after the 1st administration on the day of measurement except for the measurement on the 10-day.

Intravenous administration of Compound A ((−)-7-[(1R,2R)-2-(4,4-difluoro-3-oxooctyl)-5-oxocyclopentyl]heptanoic acid) at 30 μg/kg twice a day for days statistically significantly increased the pain threshold of the model paw compared with that of vehicle-control group.

TABLE 1
Effects of Compound A on pain threshold in
neuropathic pain model rats

| | | Pain threshold, g | | | | |
|---|---|---|---|---|---|---|
| | Dose | Pre- | During treatment | | | |
| Group | μg/kg | treatment | 2 day | 4-day | 7-day | 10-day |
| Control | — | 6.1 ± 0.2 | 5.8 ± 0.5 | 6.1 ± 0.4 | 6.0 ± 0.4 | 6.6 ± |
| | | | | | | 0.4 |
| Compound | 30 | 6.1 ± 0.2 | 6.8 ± 0.3 | 7.6 ± 0.3* | 8.1 ± 0.3** | 8.4 ± |
| A | | | | | | 0.1** |

*p < 0.05,
**p < 0.01 compared with control group

### EXAMPLE 2

According to the same manner described in Example 1, oral administration of Compound B ((E)-7-[(1R,2R)-2-(4,4-difluoro-3-oxooctyl)-5-oxocyclopentyl]hept-2-enoic acid) at 1 mg/kg twice a day for 7 days statistically significantly increased the pain threshold of the model paw compared with that of vehicle-control group.

TABLE 2
Effects of Compound B on pain threshold in
neuropathic pain model rats

| | | Pain threshold, g | | | | |
|---|---|---|---|---|---|---|
| | Dose | Pre- | During treatment | | | |
| Group | mg/kg | treatment | 2 day | 4-day | 7-day | 10-day |
| Control | — | 6.8 ± 0.2 | 6.6 ± 0.2 | 6.7 ± | 6.8 ± 0.3 | 7.3 ± 0.2 |
| | | | | 0.2 | | |
| Compound | 1 | 6.8 ± 0.2 | 7.7 ± 0.3* | 8.2 ± | 8.6 ± 0.2** | 8.2 ± 0.2* |
| B | | | | 0.2** | | |

*p < 0.05,
**p < 0.01 compared with control group

The above results indicate that Compound A and Compound B of the present invention are useful for the treatment of neuropathic pain and that the effects of the Compounds of the present invention increase by repeated administrations.

### EXAMPLE 3

Compound A and corresponding placebo were injected to the patients who have neuropathic pain around the low back and the lower limbs twice daily for 14 days. For the first 3 days, 30 μg of Compound A was administered twice daily (60 μg/day), and then 60 μg of Compound A was administer twice daily (120 μg/day) for the next 11 days.

The patient's pain-associated quality of life (QOL) was assessed using Japan Orthopedic Association Back Pain Evaluation questionnaires (JOABPEQ). Patients self-evaluated their QOL before (Pre-Treatment), 8 days (Day 8) and 15 days (Day 15) after start treatment by Compound A or placebo.

One of factors in JOABPEQ, social life function, was significantly improved in Compound A treated group, indicating that Compound A improves the QOL which impaired by the pain.

TABLE 3
Effects of Compounds A on QOL

| | Change in score of social life | |
|---|---|---|
| | function from pre-treatment | |
| Compound | Day 8 | Day 15 |
| Compound A | 2.7 ± 2.8 (N = 20) ^{a} | 3.0 ± 2.8 (N = 20) |
| Placebo | 0.5 ± 3.2 (N = 24) | 1.1 ± 2.2 (N = 20) |

^{a} p = 0.017 vs placebo,
^{b} p = 0.022 vs placebo

### EXAMPLE 4

Compound A and corresponding placebo were injected to the patients who have neuropathic pain around the low back and the lower limbs twice daily for 14 days. For the first 3 days, 30 μg of Compound A was administered twice daily (60 μg/day), and then 60 μg of Compound A was administer twice daily (120 μg/day) for the next 11 days.

The intensity of pain was assessed using Visual Analogue Scale (VAS). A VAS is usually a horizontal line, 100 mm in length, anchored by no pain and awful pain at each end. The patient marks on the line the point that they feel represents their perception of their current state. The VAS score is determined by measuring in millimeters from the left hand end of the line to the point that the patient marks. VAS score was measured before (Pre-treatment) and 15-days (Day 15) after start treatment by Compound A or placebo. When the ratio of VAS score on Day 15 to pretreatment is more than 25%, it was considered that the pain was improved and the patients who showed the pain improvement was defined as a responder.

The responder rate was significantly higher in Compound A-treated group than placebo treated group, indicating that Compound A improves the pain.

TABLE 4
Effects of Compound A on the pain

| Group | Rate of responder |
|---|---|
| Compound A | 94.4% (N = 18) * |
| Placebo | 62.5% (N = 16) |

* p = 0.035 vs placebo

What is claimed is:

1. A method for treating neuropathic pain in a mammalian subject, which comprises administering to the subject in need thereof an effective amount of a fatty acid derivative represented by the formula (I): 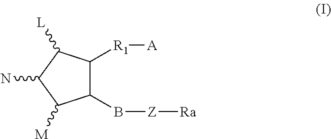
wherein L, M and N are hydrogen, hydroxy, halogen, lower alkyl, hydroxy(lower)alkyl, lower alkanoyloxy or oxo, wherein the five-membered ring may have at least one double bond;
A is —CH₃, or —CH₂OH, —COCH₂OH, —COOH or a functional derivative thereof;
B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;
Z is 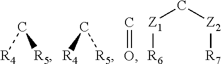
or single bond
wherein R₄ and R₅ are hydrogen, hydroxy, halogen, lower alkyl, lower alkoxy or hydroxy(lower)alkyl, wherein R₄ and R₅ are not hydroxy and lower alkoxy at the same time; Z₁ and Z₂ are oxygen, nitrogen or sulfur; R₆ and R₇ are optionally substituted lower alkyl, which is optionally linked together to form lower alkylene;
R₁ is a saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and
Ra is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group, and at least one of carbon atom in the aliphatic hydrocarbon is optionally substituted by oxygen, nitrogen or sulfur.

2. The method as described in claim 1, wherein Z is C═O.

3. The method as described in claim 1, wherein B is —CH₂—CH₂—.

4. The method as described in claim 1, wherein B is —CH₂—CH₂— and Z is C═O.

5. The method as described in claim 1, wherein L is hydroxy or oxo, M is hydrogen or hydroxy, N is hydrogen, B is —CH₂—CH₂— and Z is C═O.

6. The method as described in claim 1, wherein Ra is saturated C4-C7 (e.g. C5 or C6) aliphatic hydrocarbon residue substituted with one or more halogens (e.g. one or two halogens).

7. The method as described in claim 1, wherein R1 is a saturated or unsaturated bivalent straight or branched C5-C9(e.g. C6 or C7) aliphatic hydrocarbon residue.

8. The method as described in claim 1, wherein the fatty acid derivative is selected from the group consisting of (−)-7-[(1R,2R)-2-(4,4-difluoro-3-oxooctyl)-5-oxocyclopentyl]heptanoic acid, (E)-7-[(1R,2R)-2-(4,4-difluoro-3-oxooctyl)-5-oxocyclopentyl]hept-2-enoic acid, isomers thereof and functional derivatives thereof.

9. The method as described in claim 1, which comprises the repeated administration to the subject.

10. The method as described in claim 1, which comprises the improvement of pain-associated quality of life.
